(19) United States
(12) Patent Application Publication
(10) Pub. No.: US 2004/0260395 A1
(43) Pub. Date: Dec. 23, 2004

(54) Ophthalmological zonular stretch segment for treating presbyopia

(76) Inventor: Brian S. Boxer Wachler, Venice, CA (US)

Correspondence Address: GREENSFELDER HEMKER & GALE PC, SUITE 2000, 10 SOUTH BROADWAY, ST LOUIS, MO, 63102

(21) Appl. No.: 10/464,947

(22) Filed: Jun. 19, 2003

(51) Int. Cl. A61F 2/14

(52) U.S. Cl. 623/4.1; 623/6.37; 623/6.41

(57) ABSTRACT

The invention comprises a device for treating presbyopia. A stretch segment is provided for implantation into the region extending between the outer circumference of the lens and extending to the sulcus region at the intersection of the iris and ciliary body. The stretch segment engages the equatorial zonules spanning between the lens and ciliary body. The segment is designed to take up slack in the equatorial zonules in the presbyopic eye, such that their effective working distance is enhanced. This aids in the accommodation process which affects the curvature of the lens for near viewing. The segment may a closed ring, or may be open ended.

### BACKGROUND OF THE INVENTION

The invention relates to a method and device for treating vision problems in patients, in particular, presbyopia, otherwise known as farsightedness. With the advancement of a person's age, typically around the age of 40 years, it becomes increasingly difficult to focus one's vision on objects that are brought close to the eye. Reading print and text can especially be a problem. As is well understood in eye anatomy and physiology, this condition is caused by the failure of the lens to properly refract and focus light rays on the retina within the eye.

The refractive properties of the lens are adjusted by changing its topography, particularly its radius of curvature. The lens of the eye resides in a capsular sac, which is held in place posteriorly to the iris by a framework of collagenous fibers, known as zonules. A muscular ring of tissue to which the zonules attach, known as the ciliary body, circumferentially surrounds the lens whereby its contraction and relaxation affect the shape of the lens and therefore its refractive characteristics. The effect of the contraction of the ciliary body is to increase the refractive power of the lens by increasing the radius of curvature of the lens. This is brought about naturally when objects are brought near to the eye for close up viewing, and is known as accommodation.

The most commonly accepted theory of accommodation is that advanced by Helmholtz, which suggests that the ciliary body in its relaxed state is at its greatest distance from the circumferential bounds of the lens. At this position, it exerts a tension on the zonules which in turn stretch the lens and capsular sac, thus flattening the lens and reducing its refractive power. This is the condition for distance viewing. As the lens undergoes accommodation for near viewing, the ciliary body contracts, loosening the tension on the zonules. The natural elasticity of the capsular sac constricts the lens, causing the lens to adopt a shape having a larger radius of curvature. With a person's advanced age, however, as the Helmholtz theory goes, the elasticity of the capsular sac decreases, and the ability to effect the necessary lens curvature diminishes. Accordingly, near vision is adversely affected.

A different theory of accommodation, advanced by Schachar, departs from the view that relaxation of the zonules and a corresponding relaxation of the lens and capsular sac affects accommodation. Rather, as Schachar's theory holds, the contraction of the ciliary body exerts tension on the zonules so that they pull on the lens and capsular sac, increasing the lens equatorial diameter. This has the effect of increasing the central volume of the lens in an orientation normal to the direction of equatorial tension to increase the lens radius of curvature and refractive power. With advancing age, the zonules slacken and the ciliary body loses the ability to exert tension on the zonules to pull on the lens and capsular sac, which adversely affects accommodation. To address this situation, Schachar prescribes increasing the distance between the lens (and capsular sac) and the ciliary body to thereby increase the effective working distance of the ciliary muscle. Several methods and devices have been suggested to bring about this effect, including stretching the sclera to retract the ciliary body from the lens, and/or shortening the zonules.

Accordingly, a need exists to enhance the effective working range of the ciliary muscle, primarily by reducing slack that has developed in the zonules, to aid the eye in accommodating for near vision in a manner consistent with Schachar's theory.

### SUMMARY OF THE INVENTION

The invention provides a device for placement in the eye which has the effect of exerting tension on the zonules spanning between the lens and capsular zonules and the ciliary body. The device is comprised of a ring, or segment thereof, having a diameter of sufficient dimension to lie beyond the outer circumferential periphery of the lens such that it is in a position to engage and press against the zonules, or against the ciliary body which, in turn, can pull on the zonules. In so doing, the device effectively diminishes the slack of the zonules thus enabling the ciliary body to make efficient use of its available working distance in pulling on the lens.

There are three types of zonules comprising the framework that attaches to the outer circumference of the lens: the anterior, posterior and equatorial zonules. The anterior and posterior zonules are thought to serve primarily to support and hold the lens in place centrally behind the iris. The equatorial zonules are thought to serve a more dynamic role and serve as the primary pulling force on the lens in the accommodation process. They span between, and have insertion points on, the lens and the ciliary body. As the person ages, the equatorial zonules become slackened and lose their effectiveness as a pulling force.

The device of the present invention takes up the slack of the equatorial zonules by directly or indirectly engaging them and exerting displacing pressure in a direction transverse to the zonules' radial directional pull on the lens. One embodiment of the device may be a ring, or semi-circular, segment made of biocompatible material that is sized to fit in the region surrounding the equator of the lens of the eye, at an anterior aspect of the ciliary muscle where the equatorial zonules attach. The segment is constructed to have an outward radial resiliency so that it resists inward pressure from its contact with the zonules. As the slack in the equatorial zonules is taken up, their effective working distance is enhanced. Accordingly, the equatorial zonules are made more efficient in the accommodation process.

The device may be adapted for placement in various positions in the region beginning immediately distally proximal to the equatorial circumference of the lens and spanning to the sulcus region which defines the juncture of the ciliary body and the iris.

The above features are objects of this invention. Further objects will appear in the detailed description which follows and will be otherwise apparent to those skilled in the art.

For purpose of illustration of this invention preferred embodiments are shown and described hereinbelow in the accompanying drawing. It is to be understood that this is for the purpose of example only and that the invention is not limited thereto.

### BRIEF DESCRIPTION OF THE DRAWINGS

FIG. 1 presents a comparative view of the anatomy of the eye in partial cross-section, showing the respective shape of the lens in its normal state for distance viewing and its state of increased curvature for near viewing.

FIG. 2 presents a comparative view in cross-section of the anatomy of the eye which suffers from presbyopia, showing slackened zonules spanning from the ciliary body to the lens in the normal state for distance viewing, which fail to provide sufficient pulling tension to bring the lens into a state of increased curvature for near viewing.

FIG. 3 presents a comparative view in cross-section of the effect of the stretch segment in placing tension on the zonules within the region surrounding the lens so that they provide sufficient pulling tension to bring the lens into a state of increased curvature for near viewing.

FIG. 4 presents a comparative view in cross-section of the effect of the stretch segment when placed in an alternate region surrounding the lens.

FIG. 5 is a cross-sectional view taken along lines 5-5 of FIG. 4.

FIG. 6 is a cross-sectional view taken along lines 6-6 of FIG. 3.

FIG. 7 is a view similar to FIG. 5, except the stretch segment is open-ended.

FIG. 8 is a view of an open-ended stretch segment having ends defining apertures.

FIG. 9 is a view of an open-ended stretch segment having ends defining a hook and eyelet structure.

### DESCRIPTION OF THE INVENTION

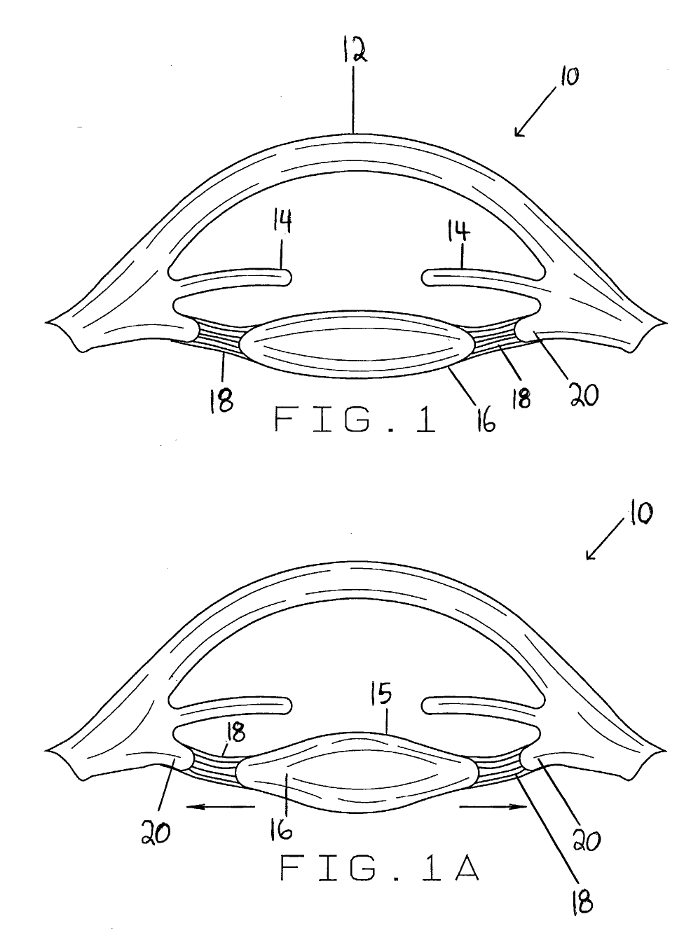

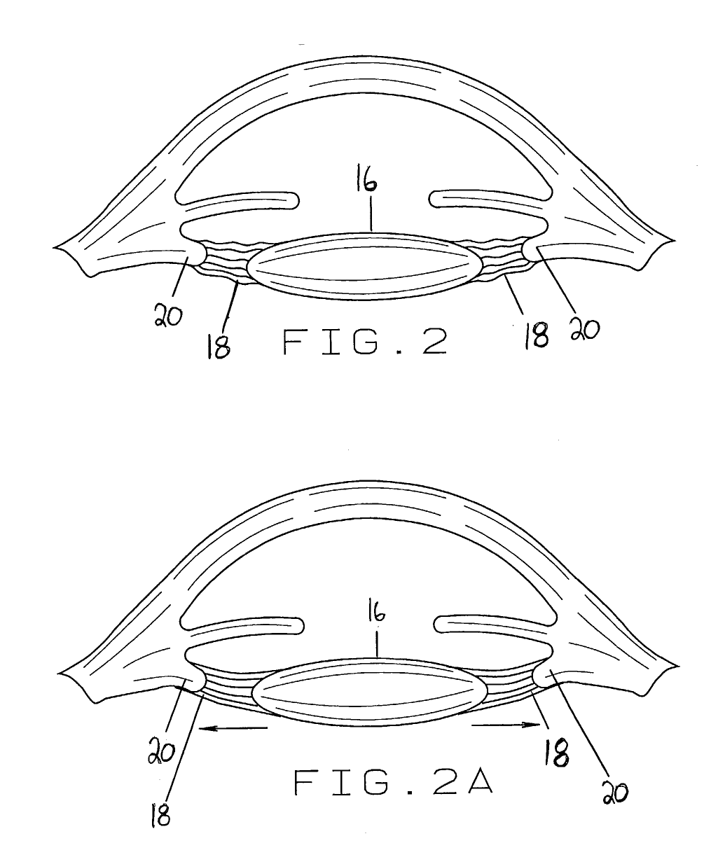

Under the theory of accommodation followed by the inventor, it is a pulling effect of the equatorial zonules that causes the change in shape necessary to refract incident light rays traveling through the eye into proper alignment on the retina. FIG. 1 diagrammatically shows the basic anatomy of the human eye 10. The cornea 12 permits the transfer of light into the eye, and the iris 14 regulates the amount of light which passes through the lens 16. A framework of radially arrayed zonules 18 support the lens in place behind the iris. The zonules span between the lens 16 and ciliary body 20. While it is understood that anterior, equatorial and posterior zonules make up the framework, for diagrammatic purposes, only the equatorial zonules are discussed. Furthermore, under the theory of accommodation followed, it is primarily the equatorial zonules that are critical in affecting the lens shape change. As the ciliary body 20 contracts, as shown in the lower drawing of FIG. 1, it exerts an outward pulling force on the zonules 18, which in turn pull outwardly on lens 16 at its periphery. This causes the central bulge 15 in the lens with a greater degree of curvature which permits the passing light rays to refract appropriately to focus on the retina. In the eye of a person having presbyopia, the spanning zonules 18 have become slack and have lost their tautness between ciliary body 20 and lens 16, as shown in FIG. 2. In such a case, the contraction of the ciliary body 20 is only effective in drawing out the slack in the zonules. The full range of the contraction does not adequately translate through the zonules, and therefore, only a minimal, if any, stretching effect is placed on lens 16. Accordingly, lens 16 does not undergo a significant change in curvature, as seen in the lower drawing of FIG. 2, and light can not refract appropriately as it passes through the lens for near viewing. Although the zonules have lost their tautness, they have not lost the ability to exert a pulling force. The zonules could again become effective in pulling and stretching the lens if their slack was reduced.

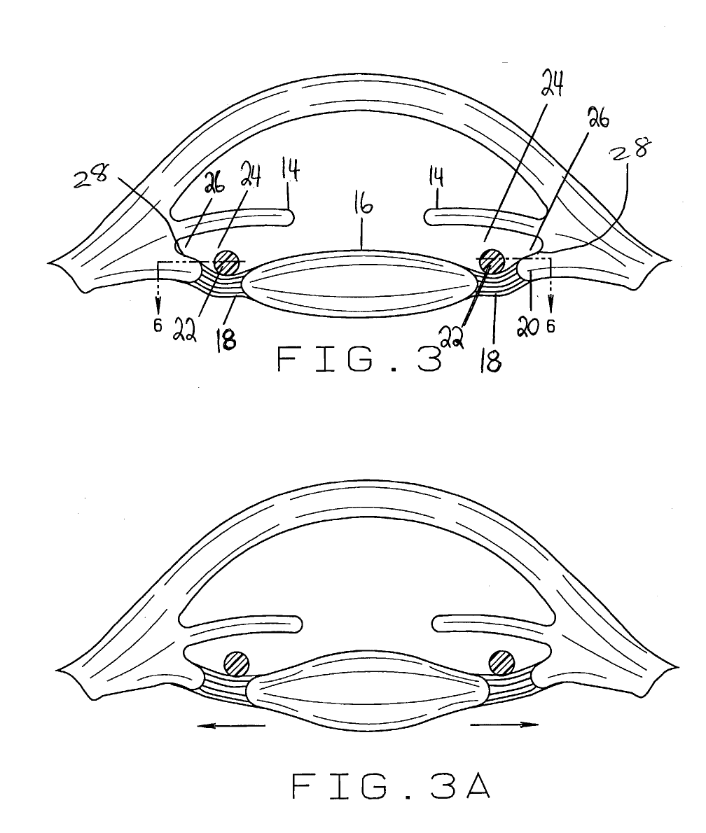

By means of the instant invention, a stretch member capable of being inserted into the region outside the equator of the lens is provided for placing tension against the equatorial zonules spanning between the lens and the ciliary body. As shown in FIG. 3, a stretch segment member 22, which may comprise a closed or open-ended ring, is inserted in the circumferential region 24 surrounding the lens 16. Segment member 22, acting as a cam, pushes against zonules 18 in a direction transverse to their length which shortens the effective length of the zonules between the ciliary body and the lens, effectively removing the slack in the zonules. By doing so, the translational effect of the contraction of the ciliary body through the zonules is increased. Accordingly, the capability of the presbyopic eye to control the shape of the lens for near viewing can be regained.

The stretch segment is preferably made of a biocompatible materials such as polymethyl methacrylate (PMMA), although those skilled in the art would recognize that other biocompatible materials suitable for implantation into the eye may be used. The diameter of the stretch segment when inserted into the eye should be in the range of 6-20 millimeters. This approximates the dimensional range available in the region 24 between the outer equatorial circumference of the lens 16 extending to the sulcus 26 defining the border between the anterior aspect 28 of ciliary body 20 and iris 14 of a typical patient. The cross-sectional dimension of the stretch segment should be in the range between 10 microns to 3 millimeters, and preferably 0.5 to 1.5 millimeters, but must have a great enough size to effectively engage and transversely displace the zonules to a sufficient degree such that slack is removed. FIGS. 3 and 6 show one embodiment of the stretch segment 22 having a diameter slightly larger than the circumference of the lens. In this embodiment, the segment resides in the gap 24 between the lens and ciliary body. Although not shown, it is to be understood that the segment may generally be disposed between the equatorial zonule bundles, and therefore, displacement may occur on either side of the segment. For diagrammatic purposes and ease of understanding the intended function of the invention, the drawings only show one side of the segment engaging the zonules.

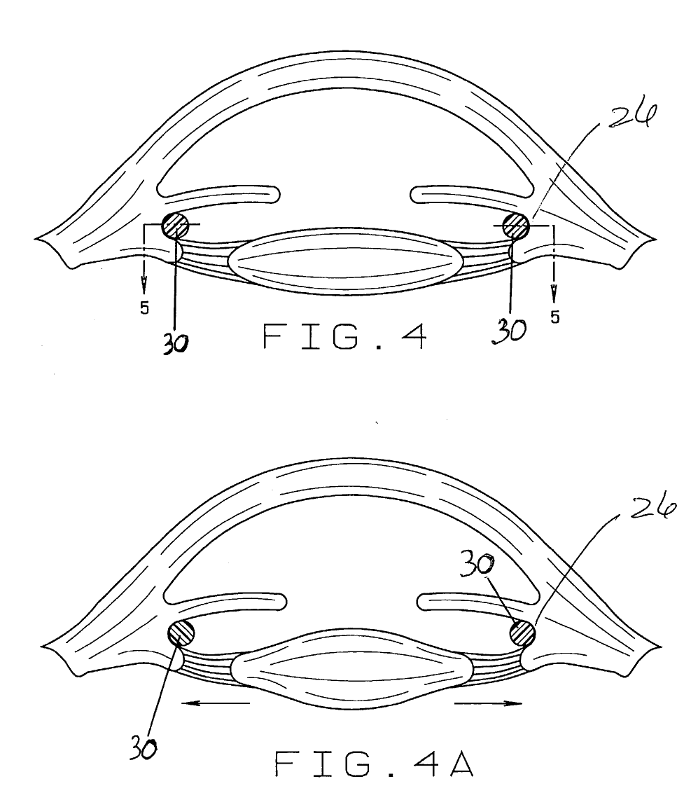

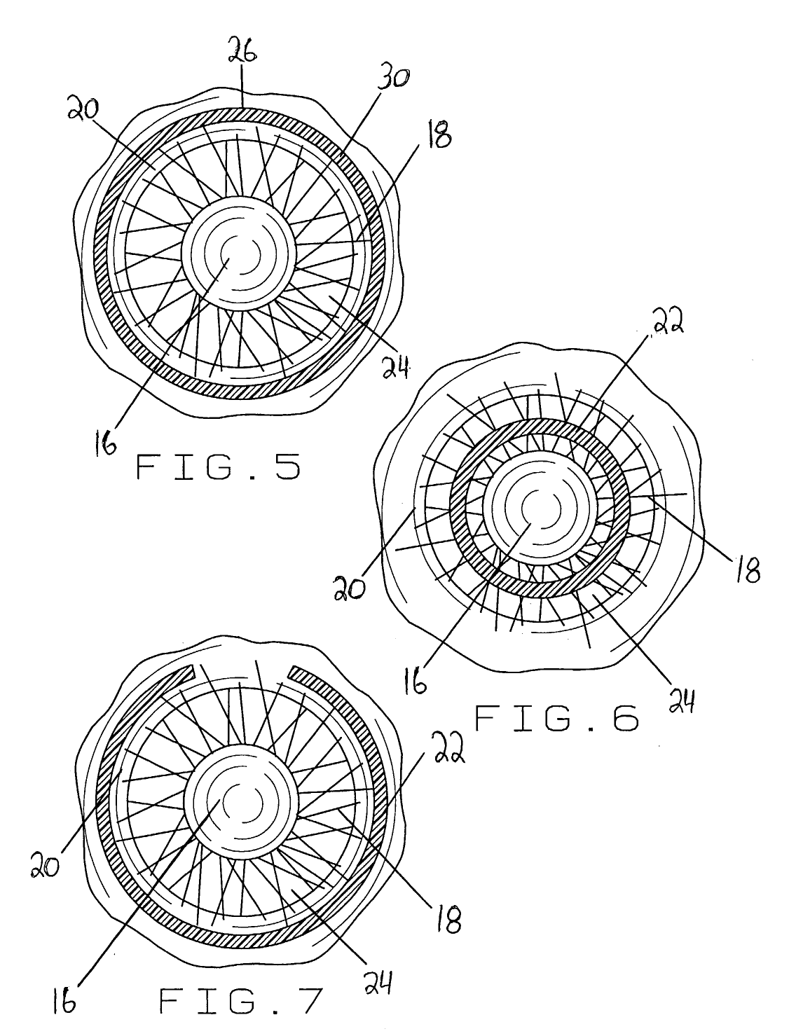

FIGS. 4 and 5 show another embodiment of a stretch segment 30 having a diameter approximating that of the sulcus 26. In this embodiment, the segment resides snugly in the sulcus. In both of these embodiments, the respective stretch segments are adapted to directly engage the equatorial zonules. In yet a further embodiment (not shown), the stretch segment has a dimension slightly larger than the sulcus 26. The segment engages the distal, anterior aspect of the ciliary body with a radially outwardly extending force which does not directly engage the zonules, but indirectly provides a supplementary stretching effect on the zonules.

One embodiment of the stretch segment may form a closed ring having a fixed diameter. Another embodiment contemplates an open, semi-circular member designed for optimal placement in the sulcus as shown in FIG. 7. The stretch segment may provide effective therapeutic benefit by engaging less than all the equatorial zonules, so long as a majority of the equatorial zonules are engaged. The segment may reach around to between 180° to approximately 360°, and more preferably to between 270° to 360° of a circle, and is constructed to have a slight resistance to flexion so that it will fit snugly into the sulcus and provide an outward force against the ciliary body. This embodiment provides for custom fitting into a patient's eye, and a segment can be constructed according to precise measurements depending on the degree of zonular stretching desired. The radius of curvature of a stretch segment can be configured to be slightly larger than the radius of the curvature of the patient's sulcus region, so that the resultant diameter of the segment will be greater than that of the sulcus for a snug fit.

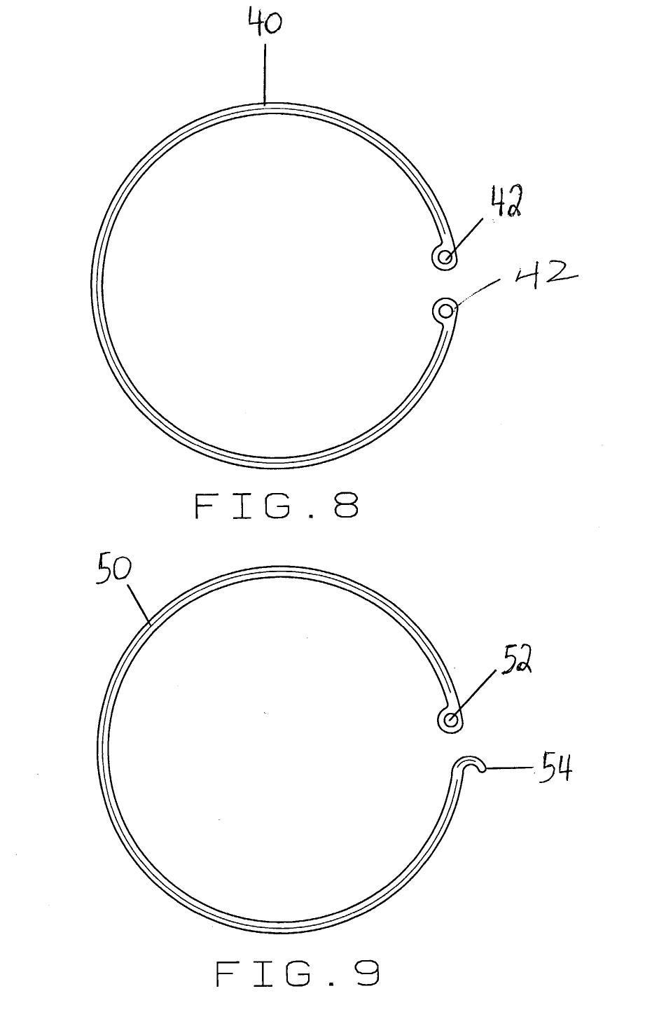

It may be necessary to secure the ends of the semi-circular stretch segment member, either together or to a part of the eye, once it is placed within the eye. FIGS. 8 and 9 show two embodiments which provide the ability to draw and secure the two ends of the stretch segment together. FIG. 8 shows stretch segment 40 having apertures 42 at each end. A fastening member, such as a suture filament, clip, or other device known to those skilled in the art, may be inserted in the apertures. The double-ended apertures provide for adjusting the effective diameter of the stretch segment as necessary. Also, the apertures permit one or both ends to be sutured to a supporting surface within the eye. FIG. 9 shows open ended stretch segment 50 having an end formed with aperture 52 and a complementary clasp 54 formed at the other end. Clasp 54 has a shape enabling it to be manipulated to pass through aperture 52 in a sideways orientation and be secured therein by a retaining flange member at the end of clasp 54. The apertures may also assist in the insertion and removal of the stretch segment.

The stretch segment may be inserted in place using techniques similar to the implantation of similar devices such as lens capsular tension rings. The stretch segment can be inserted with a forceps through a 1.0 to 4.0 mm corneal incision. Alternately, an inserting device such as that provided by Ophtec BV (EASYContro™ Micro Inserter) may be used. The Micro Inserter is a hollow tube with a hook which can engage the open end of the stretch segment. The tube is inserted through a corneal incision into the anterior chamber of the eye. The distal end of the tube is placed near the sulcus region where the segment will be implanted. The plunger holding the segment in the tube is manipulated to push the segment out of the tube, causing the segment to glide into the sulcus. The stretch segment is placed in the anterior aspect of the zonular framework to engage the front surface of the equatorial zonules.

Various changes and modifications may be made within this invention as will be apparent to those skilled in the art. Such changes and modifications are within the scope and teaching of this invention as defined in the claims appended hereto.

What is claimed is:

1. A device for treating presbyopia in humans, said device comprising a member adapted to fit in a region around an outer periphery of an equator of a lens within an eye, wherein said region is at an anterior aspect of ciliary muscle surrounding said lens at or near insertion in said ciliary muscle of equatorial zonules spanning from said lens, said member being adapted to exert a resilient force to apply tension to said equatorial zonules, said member comprising a semi-circular segment of biocompatible material, said segment having a sufficient rigidity to resist a radially inward-extending pressure exerted on said segment, said segment having a circumferential dimension greater than an outer circumferential dimension of said region.

2. The device of claim 1 in which a diameter of said member is between 6-20 millimeters.

3. The device of claim 1 in which a cross-sectional diameter of said member is between 10 microns to 3 millimeters.

4. The device of claim 1 in which said semi-circular segment is open-ended, said segment comprising greater than 180° and up to 360° of a circumference of a circle.

5. The device of claim 1 in which said member is open-ended and sufficiently flexible to be manipulated for linear insertion into a first part of said region, and able to slide around and within said periphery to circumnavigate said lens, said segment having ends adapted for connection to each other after said segment is placed in said region.

6. The device of claim 5 in which said ends define apertures, said apertures being adapted to receive a fastening member for drawing and holding said ends together.

7. A device for treating presbyopia in humans, said device comprising a member adapted to fit in a region around an outer periphery of an equator of a lens within an eye, wherein said region is at an anterior aspect of ciliary muscle surrounding said lens at or near insertion in said ciliary muscle of equatorial zonules spanning from said lens, said member being adapted to exert a resilient force to apply tension to said equatorial zonules, said member comprising a circular segment of biocompatible material, said segment having a sufficient rigidity to resist a radially inward-extending pressure exerted on said segment, said segment having a circumferential dimension greater than an outer circumferential dimension of said region.

8. The device of claim 7 in which a diameter of said member is between 6-20 millimeters.

9. The device of claim 7 in which a cross-sectional diameter of said member is between 10 microns to 3 millimeters.

10. A device for treating presbyopia in humans, said device comprising a member adapted to fit in a region around an outer periphery of an equator of a lens within an eye, wherein said region is at an anterior aspect of ciliary muscle surrounding said lens at or near insertion in said ciliary muscle of equatorial zonules spanning from said lens, said member being adapted to exert a resilient force to apply tension to said equatorial zonules, said member comprising a semi-circular segment of biocompatible material, said segment having a sufficient rigidity to resist a radially inward-extending pressure exerted on said segment, said segment having a circumferential dimension greater than an outer circumferential dimension of said outer periphery of said lens equator.

11. The device of claim 10 in which a diameter of said member is between 6-20 millimeters.

12. The device of claim 10 in which a cross-sectional diameter of said member is between 10 microns to 3 millimeters.

13. The device of claim 10 in which said semi-circular segment is open-ended, said segment comprising greater than 180° and up to 360° of a circumference of a circle.

14. The device of claim 10 in which said member is open-ended and sufficiently flexible to be manipulated for linear insertion into a first part of said region, and able to slide around and within said periphery to circumnavigate said lens, said segment having ends adapted for connection to each other after said segment is placed in said region.

15. The device of claim 14 in which said ends define apertures, said apertures being adapted to receive a fastening member for drawing and holding said ends together.

16. A device for treating presbyopia in humans, said device comprising a member adapted to fit in a region around an outer periphery of an equator of a lens within an eye, wherein said region is at an anterior aspect of ciliary muscle surrounding said lens at or near insertion in said ciliary muscle of equatorial zonules spanning from said lens, said member being adapted to exert a resilient force to apply tension to said equatorial zonules, said member comprising a circular segment of biocompatible material, said segment having a sufficient rigidity to resist a radially inward-extending pressure exerted on said segment, said segment having a circumferential dimension greater than an outer circumferential dimension of said outer periphery of said lens equator.

17. The device of claim 16 in which a diameter of said member is between 6-20 millimeters.

18. The device of claim 16 in which a cross-sectional diameter of said member is between 10 microns to 3 millimeters.
